(19) United States
(12) Patent Application Publication
(10) Pub. No.: US 2011/0223137 A1
(43) Pub. Date: Sep. 15, 2011

(54) COMPOSITIONS FOR USE IN LOW-BIRTH WEIGHT INFANTS

(75) Inventors: Dominique Darmaun, Nantes (FR); Marie-Claire Fichot, Blonay (CH); Hugues Piloquet, Bouguenais (FR); Florence Rochat, Montreux (CH); Carole Rouge, Villefranche sur Saone (FR); Jean-Christophe Roze, Nantes (FR)

(73) Assignee: NESTEC S.A., Vevey (CH)

(21) Appl. No.: 13/129,547

(22) PCT Filed: Nov. 23, 2009
(86) PCT No.: PCT/EP09/65634

§ 371 Date: May 16, 2011

(30) Foreign Application Priority Data
Dec. 5, 2008 (EP) 08170806.7

(51) Int. Cl. A61K 35/66; A61K 35/74; A61P 1/00; A61K 36/06

(52) U.S. Cl. 424/93.3; 424/93.1; 424/93.4; 424/93.41; 424/93.42; 424/93.44; 424/93.45; 424/93.46; 424/93.5; 424/93.51

(57) ABSTRACT

The present invention relates to compositions for use in low-birth weight infants. In particular, the compositions are probiotic compositions which are used for achieving full enteral feeding in low birth weight infants. The invention is also concerned with the use of specific probiotics in the manufacture of low-birth weight infant formulations.

### FIELD OF THE INVENTION

The present invention relates to compositions for use in low-birth weight infants. In particular, the compositions are probiotic compositions which are used for achieving full enteral feeding in low birth weight infants. The invention is also concerned with the use of specific probiotics in the manufacture of low-birth weight infant formulations.

### BACKGROUND OF THE INVENTION

In neonatal intensive care units, the immaturity of intestinal function, frequent use of broad-spectrum antibiotics, delay in initiating enteral feeding, infection control procedures, and pasteurisation of milk limit the exposure of preterm infants to normal commensal microorganisms. As a consequence, low birth weight (≦1,500 g) preterm infants experience a delayed and abnormal pattern of gut colonisation, particularly with regard to bifidobacteria and lactobacilli, normally dominant in healthy full term infants. This impaired intestinal colonisation may predispose preterm infants to unwanted conditions such as necrotising enterocolitis, increase in the risk of bacterial translocation, etc.

At such a stage, the immaturity of the intestinal function makes full enteral feeding quasi impossible. As a consequence, all babies with a low birth weight routinely receive intravenous nutrition for several weeks. This is however a very invasive technique associated with a high risk of complications, including catheter-related sepsis, thrombosis, and cholestasis.

Enteral supplementation of probiotics has been found to reduce the incidence of necrotising enterocolitis in very preterm infant as concluded from the trials described for example by Deshpande, G. et al. in Lancet, 2007, 369, 1614-1620, by Alfaleh, K. et al. in Cochrane Database Syst. Rev., 2008, (1), CD005496 or by Kitajima, H. et al., in Arch. Dis. Child, 1997, 76, F101-107.

Indrio, F. et al. in the Journal of Pediatrics, 2008, 801-806, also describe the effect of dietary supplementation with a probiotic on feeding tolerance and gastrointestinal motility in healthy formula-fed preterm infants. However, this study fails to address the needs of preterm infants having a birth weight of no more than 1500 g. In addition, the supplementation with probiotics did not result in increased enteral feeding volume.

Therefore, although recent reports suggest supplementation with probiotics may enhance intestinal function in low birth weight premature infants, the enteral feeding rate has not been improved.

There thus remains a need to shorten the time to reach full enteral feeding in low-birth weight infants such that intravenous nutrition can be reduced or even prevented. A putative reduction in the duration of parenteral nutrition would thus potentially benefit a very large number of low birth weight infants.

### OBJECT OF THE INVENTION

It is thus an object of the present invention to decrease the frequency and/or duration of parenteral nutrition in low birth weight infants.

### SUMMARY OF THE INVENTION

The object is achieved by means of the independent claims. The dependent claims further develop the central idea of the invention.

Thus, in a first aspect, the invention is concerned with a probiotic composition for use in achieving full enteral feeding in infants having a birth weight of no more than 1500 g.

In a further aspect, the invention also relates to the use of Lactobacillus rhamnosus and Bifidobacterium longum in the manufacture of a preterm infant formulation.

### FIGURES

The present invention is further described hereinafter with reference to the accompanying drawings in which:

FIG. 1 describes a trial profile and

FIG. 2 shows time to reach full enteral feeding. In particular it shows Kaplan-Meier curves to full enteral feeding in the two treatment groups: in all infants (top) and infants with a birth weight 1500 g.

### DETAILED DESCRIPTION OF THE PRESENT INVENTION

In the present invention, probiotic micro-organisms are considered to be micro-organisms which beneficially affect a host by improving its intestinal microbial balance (Fuller, R; 1989; J. Applied Bacteriology, 66: 365-378).

According to the present invention, it has been found that probiotic compositions can be used for achieving full enteral feeding in infants having a birth weight of no more than 1500 g.

Generally, infants having a birth weight of no more than 1500 g are preterm infants and/or infants having suffered intrauterine growth retardation.

Preterm infants are babies born before the gestational age of 37 weeks. In a particular embodiment, the present invention in particular addresses preterm infants, wherein the preterm infants have a gestational age of less than 32 weeks.

By “achieving full enteral feeding” is meant that the infants can ingest food without any adverse effects to their health. Full enteral feeding is achieved when the infants are able to absorb and digest the feeds, preferably without adverse effects such as diarrhoea and/or regurgitation. Full enteral feeding is also achieved when the feeds are able to provide the infants with all the energy and nutrients required via the gastrointestinal tract.

Thus, the composition may also be used for improving enteral feeding management in low-birth weight infants.

Full enteral feeding may be achieved by improving the gastrointestinal tolerance of said infants. This is a particular challenge in low birth weight infants who have immature digestive and motile functions and who do not have a fully colonised intestine at birth.

The composition of the invention may therefore be used for improving the tolerance of low birth weight infants to enteral feeds.

In a particular embodiment, the composition of the invention can be used for improving gastrointestinal tolerance of low-birth weight infants.

It has been surprisingly found that the compositions of the invention not only aid in achieving full enteral feeding but also have the ability to enhance the enteral feeding rate and reducing the time to full enteral feeding.

Thus, the composition of the invention can be used for reducing the length of time post-partum during which parenteral feeding is necessary.

By low birth weight infants is meant infants who weigh no more than 1500 g at birth.

The mechanism(s) by which probiotics may enhance intestinal function in very premature infant remain to be elucidated. Without wishing to be bound by theory, the mechanisms could include: decreased bacterial attachment to gut mucosa, improved intestinal barrier function, protection against ischemic injury, or a decrease in NF-kB mediated inflammatory response.

Examples of suitable probiotic micro-organisms include yeasts such as Saccharomyces, Debaromyces, Candida, Pichia and Torulopsis, moulds such as Aspergillus, Rhizopus, Mucor, and Penicillium and Torulopsis and bacteria such as the genera Bifidobacterium, Bacteroides, Clostridium, Fusobacterium, Melissococcus, Propionibacterium, Streptococcus, Enterococcus, Lactococcus, Staphylococcus, Peptostrepococcus, Bacillus, Pediococcus, Micrococcus, Leuconostoc, Weissella, Aerococcus, Oenococcus and Lactobacillus.

Specific examples of suitable probiotic microorganisms which may be used in the present invention include: Saccharomyces cereviseae, Bacillus coagulans, Bacillus licheniformis, Bacillus subtilis, Bifidobacterium bifidum, Bifidobacterium infantis, Bifidobacterium longum, Bifidobacterium lactis, Bifidobacterium breve, Enterococcus faecium, Enterococcus faecalis, Lactobacillus acidophilus, Lactobacillus alimentarius, Lactobacillus casei subsp. casei, Lactobacillus casei Shirota, Lactobacillus curvatus, Lactobacillus delbruckii subsp. lactis, Lactobacillus farciminus, Lactobacillus gasseri, Lactobacillus helveticus, Lactobacillus paracasei, Lactobacillus johnsonii, Lactobacillus reuteri, Lactobacillus rhamnosus (Lactobacillus GG), Lactobacillus sake, Lactococcus lactis, Micrococcus varians, Pediococcus acidilactici, Pediococcus pentosaceus, Pediococcus acidilactici, Pediococcus halophiles, Streptococcus faecalis, Streptococcus thermophilus, Streptococcus salivarius, Staphylococcus carnosus, and Staphylococcus xylosus.

Without wishing to be bound by theory, it is thought that Lactobacillus GG in particular may protect intestinal epithelial cells from oxidative stress by inducing the expression of heat shock chaperone protein, and activating signal transduction pathways in enterocytes.

Lactobacillus acidophilus may modulate abdominal pain through an induction of opioid and cannabinoid receptors in intestinal cells as suggested in Rousseaux, C. et al. in Nat. Med., 2007, 13, 35-37.

In the present invention, it has been found that the probiotics are preferably selected from Lactobacillus rhamnosus, Bifidobacterium longum or mixtures thereof. More preferably, the probiotics are Lactobacillus rhamnosus GG ATCC 53103 or Lactobacillus rhamnosus CGMCC 1.3724 and Bifidobacterium longum BB536 registered under ATCC BAA-999. These strains of microorganisms are available commercially.

In one embodiment the invention comprises the strains Lactobacillus Reuteri ATCC55730, Lactobacillus Reuteri DSM-17938, commercially available from Biogaia AB (Kungsbroplan 3A, Stockholm, Sweden)

The probiotics may be in a powdered, dried form. Further, if desired, the probiotic micro-organism may be encapsulated to further increase the probability of survival; for example in a sugar matrix, fat matrix or polysaccharide matrix.

The probiotic composition of the invention has been found to be useful in achieving full enteral feeding in infants having a birth weight of no more than 1500 g.

In particular, the present probiotic composition has been found to be beneficial for use in infants weighing no more than 1500 g. Preferably, the infants have a birth weight of between 1000-1500 g. Indeed, it has been found that in this weight group the benefits of the invention are achieved quicker and/or to a larger extent.

Preferably, in the present invention, the time to achieve full enteral feeding is less than 50 days, more preferably less than 40 days, most preferably less than 30 days.

The complications associated with parenteral feeding can therefore be mitigated to a quicker extent by the present composition.

The compositions of the invention can therefore be used in improving enteral feeding management in low birth weight infants.

The probiotic composition of the invention may be part of an infant oral formulation. The formulation may include components commonly used in infant formulation, in particular in low birth weight infant formulations. For instance, the formulation typically comprises fats, proteins, carbohydrates, minerals and micronutrients.

The fats may be selected from essential fatty acids, oils such as MCT oils etc. The proteins are preferably selected from dairy protein. Carbohydrates may be selected from maltodextrin, lactose etc. The micronutrients may include vitamins, etc.

The formulation may be a solution or may be in the form of a powder to be reconstituted. Such formulation may be a milk powder comprising said probiotics. Upon reconstitution, the formulation can be fed to preterm infants and thus improve their enteral feeding management.

The amount of probiotics is preferably at least 10⁷ to 10⁹ cfu per gram of composition, preferably 2×10⁸ to 8×10⁸ cfu per gram. In a preferred embodiment, it may contain 4×10⁸ cfu per gram of composition if given as a supplement to breast milk. In another embodiment, it may comprise 2×10⁸ cfu per gram if incorporated into a preterm infant formula.

It is preferably used on a daily basis. Thus, it may be used as a daily enteral supplement to parenteral feeding in low-birth weight infants. For example, it may be used from once daily up to 5 times daily, depending on the needs of the preterm infant.

It is preferable that the infant should receive about 10⁶ to 10¹⁰ cfu/day, more preferably about 10⁹ cfu/day. These amounts ensure that enough microorganisms cells reach the gastrointestinal tract of the infant to achieve the beneficial effects.

The finding of the present invention that probiotics are useful in achieving full enteral feeding in infants weighing no more than 1500 g is surprising in view of the fact that the probability for the gut to be colonised by probiotic strains diminishes with decreasing birth weight, as established by Manzoni, P. et al. in Clin. Infect. Dis., 2006, 42, 1735-1742.

This is all the more unexpected since the efficacy of oral probiotics is usually limited by the frequent use of postnatal antibiotic treatment and the frequent need to withhold enteral feeding in such low birth weight infants.

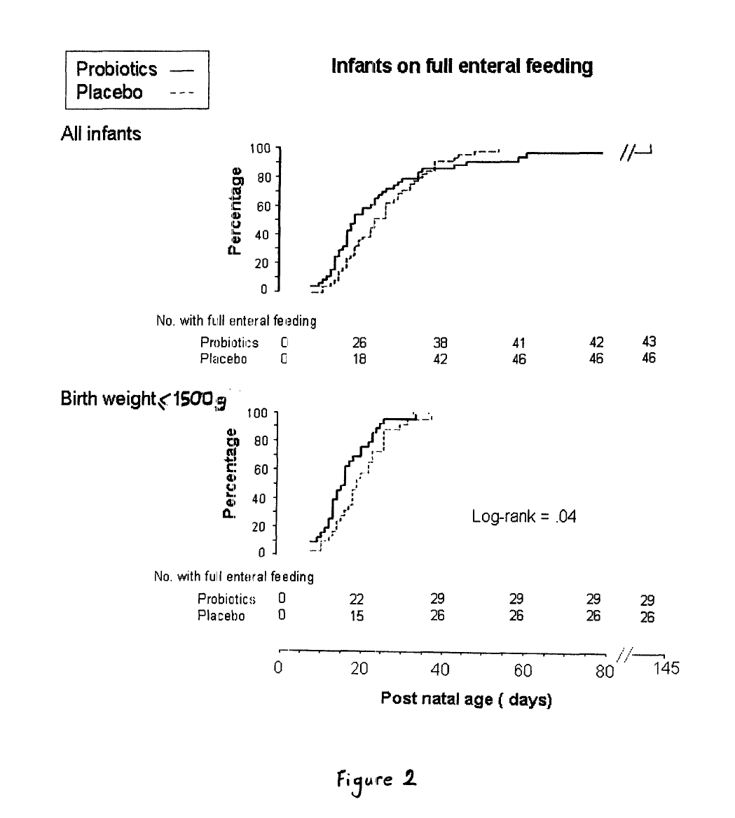

Thus, a main benefit of the invention lies in the acceleration of the switch from parenteral to enteral feeding. This provides a great relief to the infant in question by avoiding the invasive parenteral techniques or at least reducing the time during which parenteral feeding has to be carried out. FIG. 2 illustrates the difference in the time to reach full enteral feeding between infants fed a placebo composition and infants fed the composition of the invention. It further illustrates that the effect is significant in infants weighing not more than 1500 g.

The present invention, in a further aspect, also pertains to the use of Lactobacillus rhamnosus and Bifidobacterium longum in the manufacture of a low birth weight infant formulation. Preferably, the infant formulation is for infants having a birth weight of no more than 1500 g.

Preferably, the probiotic strains selected from Lactobacillus rhamnosus GG ATCC 53103, Lactobacillus rhamnosus CGMCC 1.3724, Bifidobacterium longum BB536 deposited under ATCC BAA-999 or mixtures thereof are used in the manufacture of said formulation.

The formulation may be a solution of may be in the form of a powder to be reconstituted. Such formulation could be a milk powder comprising said probiotics. Upon reconstitution, the formulation can be fed to low birth weight infants and thus improve their enteral feeding management by achieving full enteral feeding.

A method for achieving full enteral feeding in low-birth weight infants comprising the step of feeding a probiotic composition, preferably in an amount of 10⁹ cfu/day, also forms part of the invention.

The present invention is further illustrated by means of the following example.

### Example 1

### Study Population

Two centres (Mère-Enfant Hospital, Nantes, France and Institut de Puériculture, Paris, France) participated in this trial. The protocol was approved by the medical ethics committee of Nantes and is registered under the reference NCT00290576. Written, informed parental consent was obtained for each infant prior to inclusion. To be eligible for enrolment in the current study, infants had to meet the following inclusion criteria: a gestational age<32 weeks, a birth weight≦1,500 g, a postnatal age no greater than 2 weeks, the absence of any disease other than those linked to prematurity, and the enteral feeding had to have begun prior to inclusion.

### Procedure

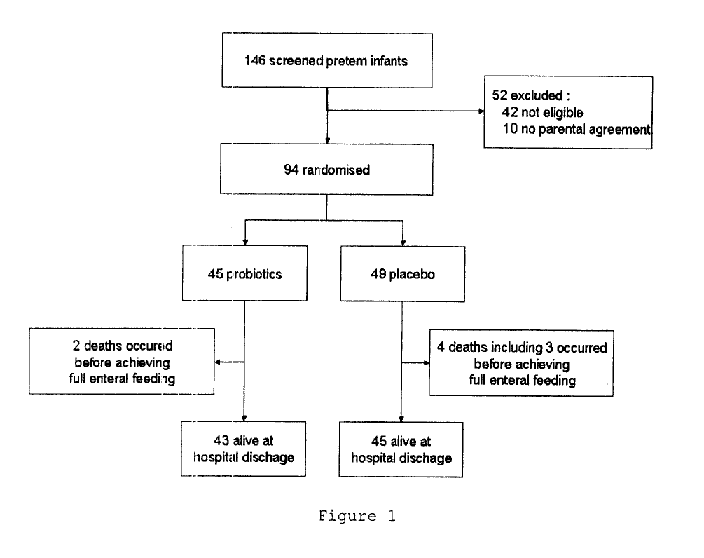

The trial profile is summarised in FIG. 1. The infants were randomised to the placebo or the probiotic group with the help of an in-house software (Nantes University Hospital, France), and randomisation was stratified on the basis of NICU (Nantes or Paris) and birth weight category (1,500 g or less, and >1500 g). Infants were fed human (own mother's expressed milk or bank milk) and/or preterm formula, and were randomly assigned to receive from the start of enteral feeding until discharge from the NICU four daily capsules of a supplement containing either (a) maltodextrin alone (referred to as placebo group), or (b) 10⁸ lyophilised cells per unit of Lactobacillus rhamnosus GG (ATCC 53103) and Bifidobacterium longum BB536 (Morinaga Milk Industry Co., Ltd., Japan) and maltodextrin (referred to as probiotic group). Placebo and probiotics prepared by Nestlé Research Center (Lausanne, Switzerland) were supplied in closed capsules and stored at 4° C. until use. Capsules were opened and mixed with 1 mL of sterile water immediately before administration to infants who received an enteral feeding on the day of the supplementation.

Stool collection was performed on the first 24 infants enrolled in each NICU for the follow-up of intestinal microbiota and faecal calprotectin. Stool samples were collected weekly from birth until hospital discharge. Intestinal microbiota was analysed weekly by culture allowing the isolation of the main genera found in preterm infants' faecal microbiota. In parallel, the dominant bacterial diversity of the intestinal microbiota was analysed by PCR-TTGE. The most prevalent molecular species were identified after sequencing by comparison of bacterial 16S rRNA gene sequences with entries in databases, using appropriate software such as BIBI®, Blast®, Multalin® and ClustalW® software. The two probiotic strains used in the present study were detected specifically in stool samples by a culture-PCR method. Faecal calprotectin concentrations were determined at 2-week intervals in duplicate using a commercial enzyme linked immunoassay (Calprest®, Eurospital, Trieste, Italy).

### Statistical Analysis

Primary outcome was the percentage of infants receiving more than half of their overall nutritional needs via the enteral route at a postnatal age of 14 days. The sample size estimation for the analysis of primary outcome was based on an expected rate of 50% in the placebo group versus 70% in the probiotic group. It was estimated that 104 patients per group were required to detect such difference with an 80% power and a 5% alpha risk. To avoid exposing an excess number of extremely premature infants to a putative risk of probiotics in the event of a potential harmful effect, a sequential trial was carried out using the Whitehead triangular test (cf. Whitehead J., Statistics in practice, 2^{nd} ed, rev. Chichester, England: John Wiley 1997). Inspection and interim analysis of data were planned every 20 patients, and performed using Pest 3.0® software. Final statistical analysis was performed using SPSS® 15.0 software. Student's t test, or Mann-Whitney when appropriate, were used for comparison of continuous variables and Khi-2 test, or Fisher's exact test when appropriate, was used for comparison of categorical variables. “Time to reach full enteral feeding” curves were computed according to the Kaplan-Meier method, and statistical comparisons were made using the log-rank test. Cox regression model was performed to adjust for the potential confounders: gestational age, center, type of enteral feeding. A logistic regression was performed to analyse whether factors were associated with the colonisation by probiotics. All tests were two-tailed. P-values less than 0.05 were considered significant.

### Results

The results of the study are shown in FIG. 2.

It can be seen that the time by which full enteral feeding is achieved is considerably diminished in preterm infant having a birth weight of no more than 1500 g.

### Example 2

| | | | per 100 kcal |
|---|---|---|---|
| ENERGY | | Kcal | 100 |
| | | kJ | 418 |
| FAT | | g | 5.01 |
| | MCT | g | 2.00 |
| | Linoleic acid | g | 0.70 |
| | alpha-Linolenic | mg | 96 |
| | acid | | |
| | Arachidonic acid | mg | 18 |
| | DHA | mg | 18 |
| PROTEIN | | g | 3.60 |
| CARBOHYDRATES | | g | 10.49 |
| | Lactose | g | 4.20 |
| | Maltodextrin | g | 6.29 |
| PROBIOTICS | Lactobacillus | cfu/g | 10⁸ |
| | rhamnosus and | | |
| | Bifidobacterium | | |
| | longum BB536 | | |
| MINERALS | | | |
| | Sodium | mg | 64.0 |
| | Potassium | mg | 137.0 |
| | Chloride | mg | 95.0 |
| | Calcium | mg | 146.0 |
| | Phosphorus | mg | 78.0 |
| | Magnesium | mg | 10.4 |
| | Manganese | μg | 14.0 |
| | Selenium | μg | 6.0 |
| MICRONUTRIENTS | | | |
| | Vitamin A | μg RE | 460 |
| | | IU | 1500 |
| | Vitamin D | μg CE | 4.7 |
| | | IU | 190 |
| | Vitamin E | mg TE | 4.0 |
| | | IU | 6 |
| | Vitamin K | μg | 8.1 |
| | Vitamin C | mg | 26.0 |
| | Vitamin B1 | mg | 0.18 |
| | Vitamin B2 | mg | 0.25 |
| | Niacin | mg | 2.00 |
| | Vitamin B6 | mg | 0.12 |
| | Folic acid | μg | 51.0 |
| | Pantothenic acid | mg | 1.00 |
| | Vitamin B12 | μg | 0.25 |
| | Biotin | μg | 4.90 |
| | Choline | mg | 25.0 |
| | Inositol | mg | 25.0 |
| | Taurine | mg | 7.9 |
| | Carnitine | mg | 3.9 |
| | Iron | mg | 2.3 |
| | Iodine | μg | 36.0 |
| | Copper | mg | 0.10 |
| | Zinc | mg | 1.50 |
| | Fluoride | μg | 6.5 |
| | Nucleotides | mg | 3.0 |

A typical composition according to the invention is shown in the table above.

1. A method for achieving full enteral feeding in infants having a birth weight of no more than 1500 g comprising administering to such an infant a composition comprising a probiotic.

2. Method according to claim 1, wherein full enteral feeding is achieved by improving the gastrointestinal tolerance of the infants.

3. Method according to claim 1, wherein the time post-partum during which parenteral feeding is necessary is reduced.

4. Method according to claim 1, wherein the time to achieve full enteral feeding is less than 50 days after birth.

5. Method according to claim 1, wherein the infant has a birth weight of between 1000-1500 g.

6. Method according to claim 1, wherein the preterm infant has a gestational age of less than 32 weeks.

7. Method according to claim 1, wherein the probiotic is selected from the group consisting of yeast, mould, bacteria and mixtures thereof.

8. Method according to claim 1, wherein the probiotics are selected from the group consisting of Lactobacillus rhamnosus, Lactobacillus Reuteri, Bifidobacterium longum and mixtures thereof.

9. Method according to claim 1, wherein the probiotics are selected from the group consisting of Lactobacillus rhamnosus GG ATCC 53103, Lactobacillus rhamnosus CGMCC 1.3724, Bifidobacterium longum BB536 deposited under ATCC BAA-999, Lactobacillus Reuteri ATCC55730, Lactobacillus Reuteri DSM-17938 and mixtures thereof.

10. Method according to claim 1, wherein the composition is part of an infant oral formulation.

11. Method according to claim 10, wherein the formulation comprises further components selected from the group consisting of carbohydrates, fats, proteins, minerals, micronutrients and mixtures thereof.

12. Method according to claim 1, wherein the amount of probiotics is at least 10⁷ to 10⁹ cfu per gram of composition.

13. Method according to claim 1, wherein the composition is fed to the infant on a daily basis.

14. Method according to claim 13, wherein the composition is fed to the infant once daily to 5 times daily.

15. Method according to claim 13, wherein the daily dose of probiotics is 10⁶ to 10¹⁰ cfu/day.

16. A method for producing an infant formula comprising the step of using Lactobacillus rhamnosus, Bifidobacterium longum, and mixtures thereof to manufacture an infant formulation.

17. Method according to claim 1, wherein the probiotics are selected from the group consisting of Saccharomyces, Debaromyces, Candida, Pichia, Torulopsis, Aspergillus, Rhizopus, Mucor, Penicillium, Torulopsis, Bifidobacterium, Bacteroides, Clostridium, Fusobacterium, Melissococcus, Propionibacterium, Streptococcus, Enterococcus, Lactococcus, Staphylococcus, Peptostrepococcus, Bacillus, Pediococcus, Micrococcus, Leuconostoc, Weissella, Aerococcus, Oenococcus, Lactobacillus and mixtures thereof.

18. Method according to claim 16, wherein the Lactobacillus rhamnosus is selected from the group consisting of Lactobacillus rhamnosus GG ATCC 53103 and Lactobacillus rhamnosus CGMCC 1.3724, and the Bifidobacterium longum is Bifidobacterium longum BB536 deposited under ATCC BAA-999.
